(19) United States
(12) Patent Application Publication
(10) Pub. No.: US 2016/0345837 A1
(43) Pub. Date: Dec. 1, 2016

(54) IMAGING APPARATUS, IMAGING METHOD, AND PROGRAM

(71) Applicant: CANON KABUSHIKI KAISHA, Tokyo (JP)

(72) Inventors: Yasufumi Takama, Kawasaki-shi (JP); Kiyohide Satoh, Kawasaki-shi (JP)

(73) Assignee: Canon Kabushiki Kaisha, Tokyo (JP)

(21) Appl. No.: 15/114,791

(22) PCT Filed: Jan. 15, 2015
(86) PCT No.: PCT/JP2015/051589

§ 371 Date: Jul. 27, 2016

(30) Foreign Application Priority Data
Jan. 29, 2014 (JP) 2014-014755
Nov. 13, 2014 (JP) 2014-230863

(51) Int. Cl. A61B 5/00; A61B 8/08; A61B 8/00; A61B 6/04; A61B 6/00

(52) U.S. Cl. CPC A61B 5/0095; A61B 5/0035; A61B 6/0414; A61B 6/502; A61B 6/5217; A61B 8/403; A61B 8/5223

(57) ABSTRACT

An image capturing condition of a target portion, such as a tumor, is adjusted while a burden of an examinee is reduced. Deformation of an MRI image obtained in advance is estimated (simulated) such that a shape of a subject in a current holding condition and a shape of the subject in the MRI image coincide with each other. Thereafter, in accordance with an obtained result of the deformation simulation, information on an image capturing condition of the target portion is estimated and displayed for a user. In this way, the user may recognize the image capturing condition in the current holding state. Consequently, risk for holding a breast by excessive holding force and risk for repetitive performance of image capturing due to inefficient holding of the target portion are reduced, and accordingly, a burden of the subject may be reduced.

### TECHNICAL FIELD

The present invention relates to an imaging apparatus, an imaging method, and a program which are suitably used for visualizing a target portion of a subject.

### BACKGROUND ART

In general, photoacoustic tomography apparatuses (hereinafter referred to as “PAT apparatuses”) have been used (refer to PTL1). Such a PAT apparatus excites absorbing substances in a subject and detects photoacoustic signals (photo-ultrasonic waves) generated due to thermoelastic expansion of the absorbing substances so as to generate an image of a feature of optical absorption of the subject. The apparatus may generate an image of distribution of optical energy accumulation amounts (distribution of optical energy absorption density) in the subject relative to irradiation light, and further generate an image of distribution of optical absorption coefficients relative to irradiation wavelengths in accordance with the image of distribution of optical energy accumulation amounts. Furthermore, the apparatus may generate an image of a state of substances constituting the subject, such as oxygen saturation of hemoglobin, in accordance with the distribution of optical absorption coefficients of a plurality of wavelengths. It is expected that such an image visualizes information on new blood vessels generated inside and outside malignant tumors, such as a cancer. Hereinafter, these images are collectively referred to as a photoacoustic tomography image (a PAT image).

Irradiated near infrared light and photoacoustic waves generated due to the irradiated near infrared light attenuate in a living body, and therefore, it is difficult for PAT apparatuses to generate an image of a deep portion of a subject when compared with imaging apparatuses using X rays. Similarly, also in ultrasonic diagnostic apparatuses which irradiate ultrasonic waves to a subject and receive reflected waves (echoes), quality of an image of a deep portion of the subject is deteriorated. Therefore, image capturing of a subject by imaging apparatuses in a state in which the subject is held such that a thickness of the subject is efficiently small is attempted. In PTL 1, a method for capturing an image in a state in which a breast is held by two plates (hereinafter referred to as “holding plates”) so that a thickness of the breast is reduced is disclosed as an embodiment of a PAT apparatus using the breast as a subject. Furthermore, in PTL2, a method for adjusting a thickness of a breast by adjusting contact pressure between the breast and a stretch film serving as a holding member is disclosed as an embodiment of an ultrasonic diagnostic apparatus using the breast as a subject. As modality of image capturing similarly performed in a state in which a thickness of a breast is reduced, an X-ray mammography apparatus may be taken as an example. In an X-ray mammography apparatus, image capturing is performed in a state in which a thickness of a breast is reduced so that a radiological dosage is reduced as much as possible by reducing overlapping between a mammary tissue and a tumor portion (refer to PTL 3).

Furthermore, PTL 4 discloses a technique of performing, when diagnosis of a breast cancer is performed using images of a subject by a plurality of modalities, positioning between the images taking deformation into consideration so that a doctor efficiently performs the diagnosis.

### CITATION LIST

### Patent Literature

PTL 1 U.S. Pat. No. 5,840,023

PTL 2 Japanese Patent Laid-Open No. 2007-282960

PTL 3 Japanese Patent No. 3431202

PTL 4 Japanese Patent Laid-Open No. 2011-224211

### SUMMARY OF INVENTION

### Technical Problem

Although the imaging apparatuses disclosed in PTLs 1 and 2 may adjust a thickness of a subject, the imaging apparatuses may not make a determination as to whether images of a target portion suitable for diagnosis have been obtained until the image capturing is terminated.

Furthermore, the imaging apparatus disclosed in PTL 3 includes a unit which adjusts pressure so that a breast is not excessively pressed. However, a determination as to whether an image suitable for diagnosis may be obtained while a breast is prevented from being excessively pressed is not instantly performed, and therefore, a period of time in which the breast is held may be increased. Moreover, PTL 4 discloses a method for estimating deformation of a subject in different three-dimensional images and estimating a position of a tumor after the deformation but does not disclose estimation of a distance between the tumor and a holding plate. Therefore, a determination as to whether images suitable for diagnosis are obtained may not be instantly performed.

The present invention provides a technique of obtaining a state of image capturing of a target portion included in a subject.

### Solution to Problem

An imaging apparatus according to the present invention includes an image obtaining unit configured to obtain a three-dimensional image of a subject, a target portion obtaining unit configured to obtain positional information of a target portion of the three-dimensional image obtained by the image obtaining unit, a condition obtaining unit configured to obtain a holding condition for holding the subject by a holding member, a deformation estimation unit configured to estimate deformation of the subject in the three-dimensional image in accordance with the holding condition obtained by the condition obtaining unit, and a state estimation unit configured to estimate a state of image capturing of the target portion in accordance with the positional information of the target portion obtained by the target portion obtaining unit and the deformation of the subject estimated by the deformation estimation unit.

### Advantageous Effects of Invention

According to the present invention, a state of image capturing of a target portion included in a subject may be obtained.

### BRIEF DESCRIPTION OF DRAWINGS

FIG. 1 is a block diagram illustrating a functional configuration of a PAT apparatus according to an embodiment of the present invention.

FIG. 2 is a diagram illustrating a method for capturing an image of a subject using the PAT apparatus.

FIG. 3 is a flowchart illustrating a procedure of a process of estimating a holding state according to a first embodiment of the present invention.

FIG. 4A is a diagram schematically illustrating the relationship between a holding condition and a holding state in a case where a breast is viewed from a front side.

FIG. 4B is a diagram schematically illustrating the relationship between a holding condition and a holding state in the case where the breast is viewed from the front side.

FIG. 5A is a diagram illustrating a deformation simulation of an MRI image.

FIG. 5B is a diagram illustrating the deformation simulation of the MRI image.

FIG. 6 is a diagram illustrating a screen displayed in a case where a distance between a holding plate and a target portion is displayed as a holding state according to the first embodiment of the present invention.

FIG. 7 is a flowchart illustrating a procedure of a process of estimating a holding state according to a second embodiment of the present invention.

FIG. 8 is a diagram illustrating a screen displayed in a case where a distance between a holding plate and a target portion is displayed as a holding state according to the second embodiment of the present invention.

FIG. 9 is a diagram illustrating a method for holding a subject according to a third embodiment of the present invention.

FIG. 10A is a diagram illustrating a holding condition according to the third embodiment of the present invention.

FIG. 10B is a diagram illustrating another holding condition according to the third embodiment of the present invention.

FIG. 11 is a diagram illustrating a method for holding a subject according to a fourth embodiment of the present invention.

FIG. 12 is a flowchart illustrating a procedure of a process of estimating a state of image capturing according to a fifth embodiment of the present invention.

FIG. 13 is a diagram illustrating a screen displayed in a case where an attenuation rate of a photoacoustic signal is displayed as the state of image capturing according to the fifth embodiment of the present invention.

FIG. 14 is a flowchart illustrating a procedure of a process of calculating recommendation values of a holding condition and an image capturing condition according to a sixth embodiment of the present invention.

FIG. 15 is a diagram illustrating a screen displayed in a case where, when the number of times irradiation is performed is set as the image capturing condition, an amount of reached irradiation light is displayed as a state of image capturing.

FIG. 16 is a diagram illustrating a screen displayed in a case where the state of image capturing has reached a target value by adjusting the holding condition and the image capturing condition according to the sixth embodiment of the present invention.

### DESCRIPTION OF EMBODIMENTS

### First Embodiment

A PAT apparatus which is an example of an imaging apparatus according to a first embodiment sets a breast as a subject, estimates a current holding state of a target portion as a state of image capturing of the target portion, such as a tumor, in accordance with a current holding condition of the subject, and displays the holding state for a user. Specifically, the PAT apparatus estimates (simulates) deformation of a three-dimensional image obtained in advance such that a shape of a subject in the current holding condition and a shape of the subject in the three-dimensional image coincide with each other. Then, the PAT apparatus estimates information on a state of image capturing of the target portion (a holding state in this embodiment) in accordance with a result of the estimation of the deformation and displays the information for the user. It is assumed here that the holding condition includes a distance (a holding length) between two holding plates which hold the subject and holding force of the holding plates for holding the subject, for example. Furthermore, it is assumed that the holding state includes a distance from one of the holding plates to the target portion (a holding-plate-to-target-portion distance) and stress applied to the target portion, for example. By this, the user may adjust the holding condition in accordance with the displayed information. The PAT apparatus according to this embodiment will now be described.

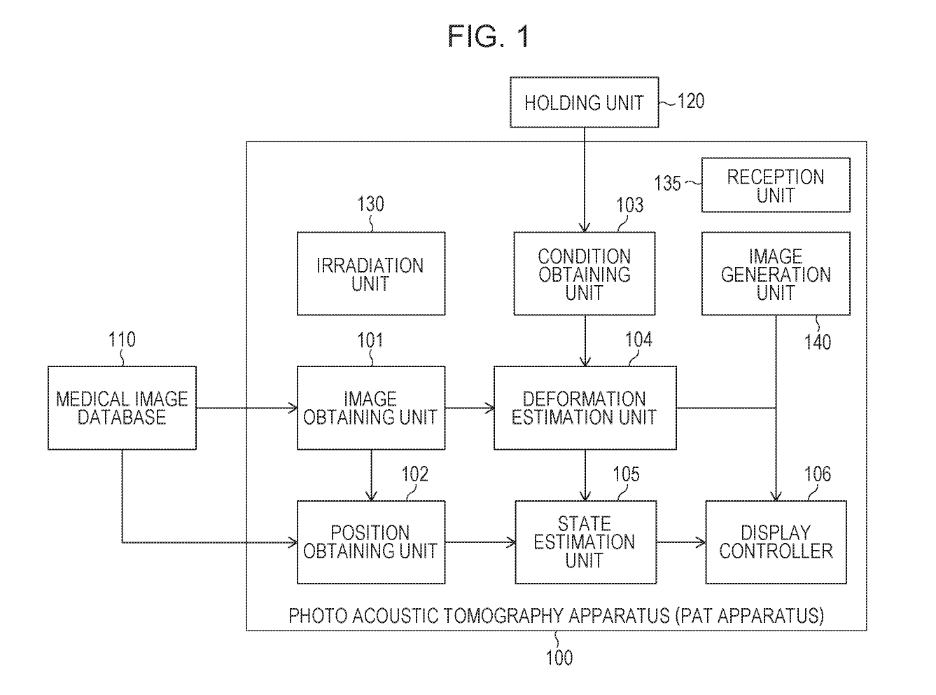

FIG. 1 is a block diagram illustrating a functional configuration of a PAT apparatus 100 according to this embodiment.

As illustrated in FIG. 1, the PAT apparatus 100 of this embodiment includes a holding unit 120, an irradiation unit 130, a reception unit 135, and an image generation unit 140. The PAT apparatus 100 further includes an image obtaining unit 101, a position obtaining unit 102, a condition obtaining unit 103, a deformation estimation unit 104, a state estimation unit 105, and a display controller 106. The PAT apparatus 100 is connected to a medical image database 110 which stores data on an MRI image as a three-dimensional image obtained by capturing an image of a breast which is a subject. The medical image database 110 may store information on a position of a target portion included in the MRI image. As the information on a position of a target portion, information on a position (a three-dimensional coordinate) of a center of gravity of a lesion region in the MRI image, information on the lesion region (such as a three-dimensional label image obtained by assigning a predetermined value to a voxel representing the lesion), and the like may be stored. Although a case where an MRI image is used as a three-dimensional image of a subject will be described below, a three-dimensional image may be an image of a subject captured by modality other than the MRI, such as an X-ray CT image, a PET image, or a three-dimensional ultrasonic tomography image. Alternatively, a PAT image obtained by capturing an image of a subject by the PAT apparatus 100 in the past may be used.

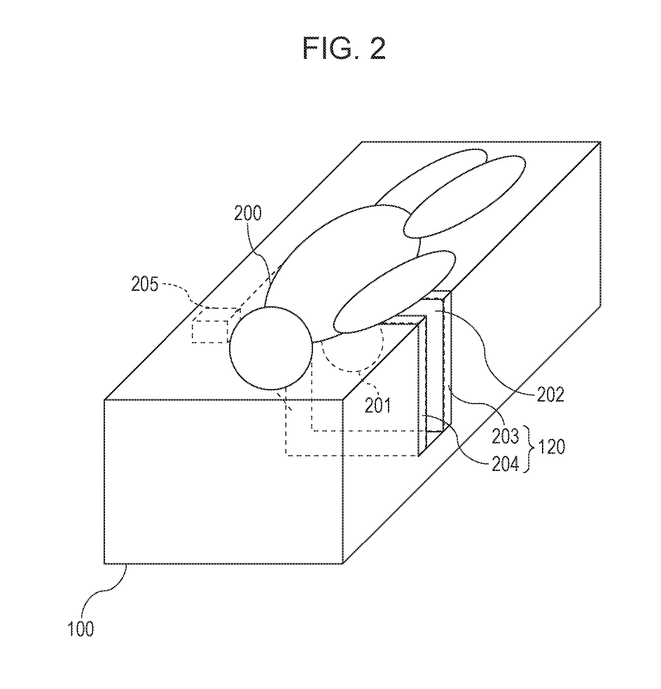

Next, a method for capturing an image of a subject employed in the PAT apparatus 100 will be described with reference to FIG. 2. In FIG. 2, an examinee 200 lies face down on a bed disposed on an upper surface of the PAT apparatus 100. Then a breast 201 which is a subject is inserted into an opening portion 202 formed on the upper surface of the PAT apparatus 100. Here, the breast 201 is held in a state in which the breast 201 is pressed by the holding unit 120 including holding members, that is, two transparent holding plates (a foot-side holding plate 203 and a head-side holding plate 204) and image capturing is performed in a state in which a thickness of the breast 201 is reduced so that irradiation light reaches an inside of the breast 201. The movable head-side holding plate 204 is moved toward the fixed foot-side holding plate 203 so that the breast 201 is held.

Furthermore, it is assumed in this embodiment that the foot-side holding plate 203 and the head-side holding plate 204 are flat plates and holding surfaces which are in contact with the breast 201 are flat. Furthermore, when the breast 201 is to be held, the condition obtaining unit 103 measures a holding condition of the breast 201. In this embodiment, the condition obtaining unit 103 measures a holding length (a distance between the foot-side holding plate 203 and the head-side holding plate 204) and holding force (force of the foot-side holding plate 203 and the head-side holding plate 204 for holding the breast 201) as holding conditions.

A near infrared light pulse is irradiated by the irradiation unit 130 from a light source, not illustrated, in a direction orthogonal to the flat planes of the foot-side holding plate 203 and the head-side holding plate 204. A photoacoustic signal generated in a body is received by an ultrasonic probe (the reception unit 135), not illustrated, which is disposed on the foot-side holding plate 203 side so as to orthogonal to a holding surface, and the image generation unit 140 executes reconfiguration of an image. As illustrated in FIG. 2, the PAT apparatus 100 includes a breast position setting camera 205 which captures an image of a state of the subject and which is disposed in a position in which the breast position setting camera 205 may capture an image of an appearance of the subject from a side. The user determines a position of the breast position setting camera 205 such that an image of the breast 201 is appropriately captured while an image (a breast position setting camera image) captured by the breast position setting camera 205 is checked, and issues an instruction for generating a PAT image.

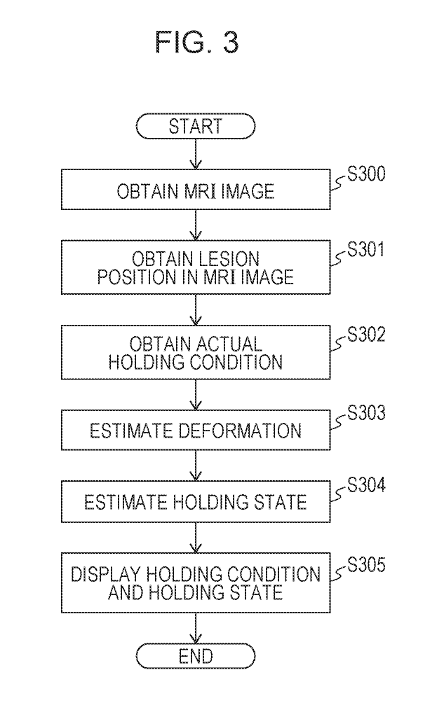

FIG. 3 is a flowchart illustrating a procedure of a process of estimating a holding state by the PAT apparatus 100 of this embodiment.

### [Step S300: Obtain MRI Image]

First, in step S300, the image obtaining unit 101 obtains an MRI image of the subject stored in the medical image database 110. The obtained MRI image is output to the position obtaining unit 102 and the deformation estimation unit 104.

[Step S301: Obtain Position of Target Portion from MRI Image]

Next, in step S301, the position obtaining unit 102 obtains information on a position of a target portion included in the MRI image obtained in step S300. The obtained positional information is output to the state estimation unit 105. The position of the target portion is obtained by manually specifying the position using a mouse, not illustrated, while the user monitors the MRI image so that a coordinate value of the position in the MRI image is obtained, for example. In a case where the positional information of the target portion has been stored in the medical image database 110, the information may be obtained. For example, information on a lesion region may be obtained from the medical image database 110 and a coordinate of the center of gravity of the region may be determined as the position of the target portion.

### [Step S302: Obtain Holding Condition]

Subsequently, the condition obtaining unit 103 measures holding conditions (a holding length and holding force) of the breast 201 which is held by the foot-side holding plate 203 and the head-side holding plate 204 in step S302. Information on the holding conditions is output to the deformation estimation unit 104 and the display controller 106.

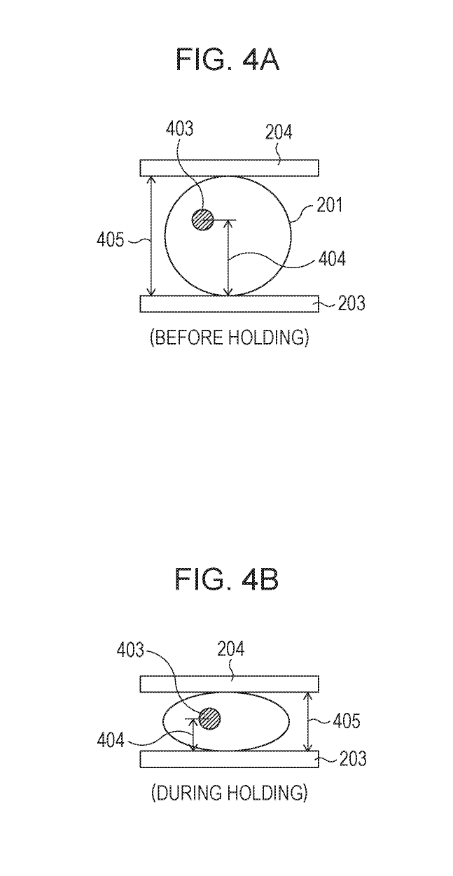

FIGS. 4A and 4B are diagrams schematically illustrating the relationships between holding conditions and holding states in a case where the breast 201 is viewed from a front side. The foot-side holding plate 203 is disposed on a side in which the probe for receiving a photoacoustic signal is disposed. A target portion 403 is a tumor in the breast 201. A holding-plate-to-target-portion distance 404 represents a distance between the target portion 403 and the foot-side holding plate 203 and is a holding state of this embodiment. As the distance 404 is smaller, attenuation of a signal generated by the target portion 403 may be suppressed, and accordingly, a PAT image suitable for diagnosis may be generated. A holding length 405 represents a distance between the movable head-side holding plate 204 and the fixed foot-side holding plate 203. The holding length 405 and holding force of the two holding plates 203 and 204 for holding the breast 201 are measured by the condition obtaining unit 103 and output as current holding conditions.

### [Step S303: Deformation Estimation]

In step S303, the deformation estimation unit 104 estimates deformation of a breast region in the MRI image obtained in step S300 in accordance with the holding conditions obtained in step S302 (simulation of pressure deformation) and outputs a result of the estimation to the state estimation unit 105.

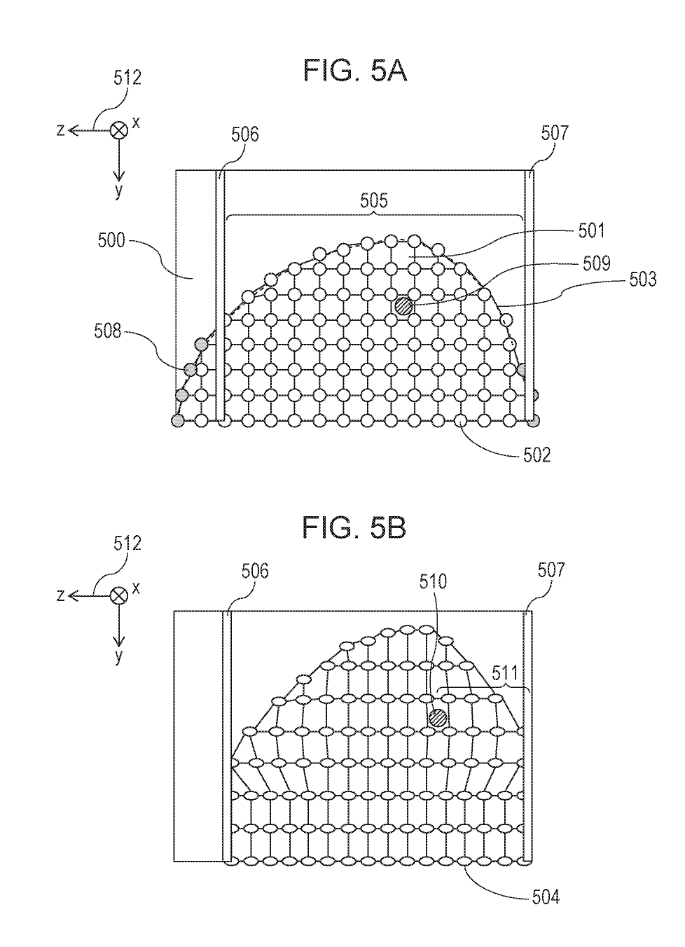

FIG. 5A is a diagram illustrating a method for estimating deformation of the breast 201 caused by the holding plates 203 and the 204 (a method for simulating pressure deformation in the MRI image). First, a three-dimensional mesh 502 is generated in a breast region 501 in an MRI image 500 obtained by the image obtaining unit 101. The breast region 501 may be extracted by detecting a body surface 503 serving as a boundary between an outside and an inside of the body of the subject from the MRI image 500 using luminance gradient of the image and determining a region in the inside of the body as the breast region 501. Then nodes and elements are disposed in the extracted breast region 501 at regular intervals so that the mesh 502 is generated. The mesh 502 may be constituted by the tetrahedral elements or hexahedral elements and the nodes which define shapes of the elements. Each of the nodes has a coordinate in an MRI image coordinate system 512, displacement of a position of the node, and information on stress. A method for generating the mesh 502 in the breast region 501 is not limited to this and any one of general methods may be used.

When a simulation of pressure deformation caused by the holding plates is performed on the generated mesh 502 using a finite element method, a deformation mesh 504 illustrated in FIG. 5B may be obtained. In actual pressure deformation caused by the holding plates, when the movable head-side holding plate 204 is moved toward the center of the subject, surface regions of the subject intruding to outsides of the holding plates after the movement are stuck to the holding plates.

Therefore, a holding plate 506 is moved such that the holding length obtained by the condition obtaining unit 103 coincides with a holding length 505 defined in the MRI image 500. Then nodes intruding to the outsides of the holding plates (outer surface nodes 508) are obtained using nodes positioned on the body surface 503. Furthermore, displacement amounts of the outer surface nodes 508 which causes the outer surface nodes 508 to be in contact with the holding plate 506 are obtained. A calculation of the finite element method is executed by assigning the displacement amounts as boundary conditions of a simulation, and accordingly, the mesh 504 obtained by deforming the breast 201 so that a current holding length is obtained is generated. In this embodiment, a period of time in which the holding plate 506 is moved such that the holding length 505 of the holding plates defined in the MRI image 500 coincides with the holding length obtained in step S302 is divided into n deformation simulations, and accordingly, change of the boundary conditions which occurs in a deformation process is addressed.

### [Step S304: Estimate Holding State]

Next, in step S304, the state estimation unit 105 estimates information on a holding state of the target portion under the current holding conditions in accordance with a result of the deformation estimation of the MRI image calculated in step S303. Specifically, as information on the holding state, the holding-plate-to-target-portion distance under the current holding conditions and stress applied to the target portion under the current holding conditions are estimated. Furthermore, the determination unit, not illustrated, included in the state estimation unit 105 compares the estimated holding state with a target value of the holding state stored in a memory, not illustrated, so as to determine whether the estimated holding state satisfies the target value (a quality determination). Then a result of the comparison is output to the display controller 106. Note that the target value may be determined in advance as a value (a reference value or an appropriate value) recommended by the PAT apparatus 100. Furthermore, the user may specify the target value through a UI, not illustrated.

Here, a method for estimating a position of the target portion under the current holding conditions in accordance with the result of the deformation estimation will be described. The state estimation unit 105 estimates a position of a target portion 510 after the pressure deformation illustrated in FIG. 5B using a position of a target portion 509 of the MRI image 500 illustrated in FIG. 5A in accordance with the result of the deformation estimation.

First, in the mesh 502 before deformation, a mesh element including the target portion 509 is searched for. Next, information on deformation of the target portion is calculated in accordance with information on displacement of all nodes which define the element. A position after the deformation may be calculated from the position before deformation using the displacement information. Subsequently, a smallest distance between the position of the target portion 510 after deformation and a fixed holding plate 507 is calculated so that a holding-plate-to-target-portion distance 511 is obtained. Furthermore, stress in the position of the target portion may be calculated by a general method in accordance with stress applied to nodes which define the element including the target portion.

### [Step S305: Display Holding Conditions and Holding State]

In step S305, the display controller 106 displays information on the current holding conditions of the subject measured in step S302, information on the holding state of the target portion estimated in step S304, and the breast-position-setting-camera image in a display, not illustrated. Furthermore, the display controller 106 displays a result of a quality determination of the holding state estimated in step S304 for the user where appropriate. For example, when the holding state does not satisfy the target value, alert information is displayed (for example, an icon representing alert is displayed beside the information on the holding state displayed in the display or a buzzer is sounded). Alternatively, display modes of the holding state are switched from one to another between a case where the holding state satisfies the target value and a case where the holding state does not satisfy the target value. For example, a color of characters or a color of a background is changed when the holding state is displayed in accordance with a result of the quality determination.

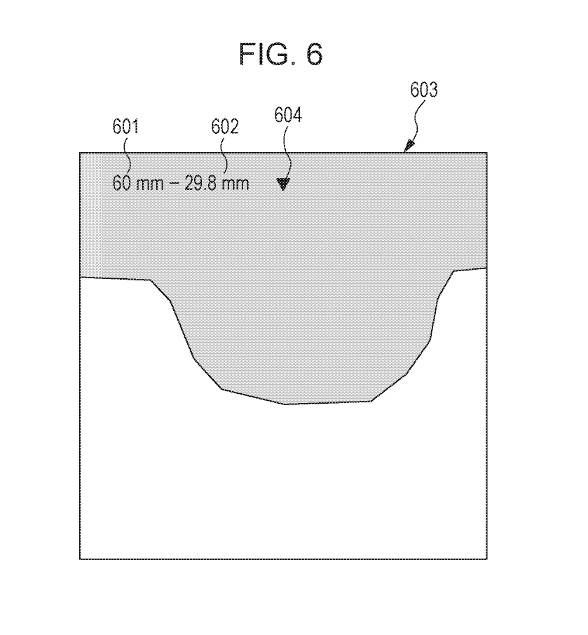

FIG. 6 is a diagram illustrating a screen displayed in a case where the holding length is displayed as the holding condition and the holding-plate-to-target-portion distance is displayed as the holding state of this embodiment. A holding length 601 illustrated in FIG. 6 corresponds to the current holding length measured in step S302. A holding-plate-to-target-portion distance 602 corresponds to the holding-plate-to-target-portion distance estimated in step S304. A breast-position-setting-camera image 603 is captured by the breast position setting camera 205. According to the example of FIG. 6, the current holding length is 60 mm and the holding-plate-to-target-portion distance is estimated as 29.8 mm. Furthermore, a “black down-pointing triangle:▾” is displayed beside the holding-plate-and-target-portion distance 602 as an icon 604 which warns that the value of the holding-plate-to-target-portion distance 602 is smaller than the target value. The user adjusts the holding condition while determining whether the holding state is appropriate in accordance with the displayed information before a PAT image is captured.

As described above, the imaging apparatus according to the present invention includes an image obtaining unit (the image obtaining unit 101) which obtains a three-dimensional image of a subject, a target portion obtaining unit (the position obtaining unit 102) which obtains information on a position of a target portion of the three-dimensional image obtained by the image obtaining unit, a condition obtaining unit (the condition obtaining unit 103) which obtains a condition for holding the subject using holding members, a deformation estimation unit (the deformation estimation unit 104) which estimates deformation of the subject in the three-dimensional image in accordance with the holding condition obtained by the condition obtaining unit, and a state estimation unit (the state estimation unit 105) which estimates a state of the image capturing of the target portion in accordance with the information on the position of the target portion obtained by the target portion obtaining unit and the deformation of the subject estimated by the deformation estimation unit. Specifically, the PAT apparatus according to this embodiment performs a deformation simulation on an MRI image of a breast in accordance with a current holding condition, estimates a holding state in accordance with a result of the simulation, and displays the estimated holding state to the user. By this, the user may recognize the holding state of the target portion under the current holding condition before the PAT image is captured. As a result, risk for holding a breast by excessive holding force and risk for repetitive performance of image capturing due to a state of inefficient holding of the target portion are reduced, and accordingly, a burden of the subject may be reduced.

Note that, although the case where the distance between the target portion and the fixed holding plate (the foot-side holding plate 203) is estimated as a holding state is illustrated in this embodiment, information on any holding state other than the holding state of the distance may be estimated as long as the information on a holding state of image capturing of the target portion is estimated. For example, a distance between a position where irradiation light enters and the target portion may be estimated as the holding state. For example, when irradiation light enters on the head-side holding plate 204 side, a distance between the head-side holding plate 204 to the target portion may be estimated by a calculation the same as that performed in step S304 and information on the distance may be displayed. The distance between the position where the irradiation light enters and the target portion is preferably short since the irradiation light attenuates in the body, and this information is effective as reference information used by the user to adjust the holding state. Note that this embodiment which estimates the holding state of the PAT apparatus is applicable to other apparatuses which capture an image in a state in which a subject is held. For example, this embodiment is similarly applicable to estimation of a holding state in ultrasonic diagnosis apparatuses which irradiate an ultrasonic wave to a subject and receive a reflection wave (echo) of the ultrasonic wave and X-ray mammography apparatuses.

### [Modification 1-1]

The holding plates of the foregoing embodiment have flat surfaces in the foregoing embodiment. However, a shape of the holding plates is not limited to this. The holding plates may have any general shape including an arch shape and a basin shape. In this case, the deformation simulation of the subject may be performed using the general shape. Furthermore, when holding plates of a plurality of shapes are replaceable (selectable) as a holding unit, a unit which obtains information on a type of holding plate as a holding condition is provided and a simulation may be performed by selecting a shape in accordance with obtained information.

### [Modification 1-2]

In the foregoing embodiment, a method for sandwiching a breast in a horizontal direction or a vertical direction has been described as a method for holding a breast serving as a subject. However, the subject holding method is not limited to this and other holding methods may be employed. For example, when an image of a breast is to be captured, a holding method for holding a breast by a holding plate from a front (in a direction from a nipple) so that a thickness is adjusted by a pressure between the holding plate and a breast wall may be employed.

### [Modification 1-3]

In the foregoing embodiment, a shape of the holding plates is recognized in advance. However, it is not necessarily the case that the shape of the holding plates is recognized in advance, and a shape of holding plates may not be predictable or a subject may be held by non-rigid (that is, deformable) holding members. For example, a subject may be held by a holding film, such as a gum film. In this case, a shape of a body surface of the held subject is measured and a deformation simulation is performed on the subject using the shape. Here, the measurement of the shape of the body surface may be performed by a range sensor employing a time-of-flight method, for example. In this case, the measured shape of the body surface corresponds to a holding condition. Furthermore, the measurement may be performed by a stereo image process using a plurality of cameras. When the holding film is used, the holding condition may be adjusted by tension of the film.

### [Modification 1-4]

In the foregoing embodiment, the case where the distance between one of the holding plates and the target portion is displayed as a holding state provided that the subject is substantially in contact with the holding plates. However, when a gel-like matching member or matching liquid exists between the holding members and the subject, a distance between a body surface of the subject and the target portion is preferably displayed as a holding state instead of the distance from the holding plate. In this case, a shape of the body surface of the subject is measured by a range sensor or the like, and a deformation simulation is performed on the subject using the shape using information on a result of the measurement as a holding condition. Then instead of the distance from the holding member, a distance between the body surface of the subject (in particular, an incident position of light or an emission position of a sound wave) and the target portion is preferably calculated and displayed.

### [Modification 1-5]

It is not necessarily the case that information displayed as a holding state is a value of the holding state (the distance between the holding plate and the target portion), and information on a rate relative to the target value (a reaching rate) may be displayed as a holding state.

### Second Embodiment

The PAT apparatus according to the first embodiment estimates the holding state of the target portion under the current holding condition and displays information on the holding state. However, in this embodiment, holding states of a target portion under a plurality of assumed holding conditions are estimated in advance. Then an indication of the holding conditions is calculated in accordance with a target value of the holding states and the indication is displayed for a user. With this configuration, since a calculation of a deformation estimation may be eliminated while a subject is actually held, a period of time required for holding a breast corresponds to a period of time required for performing actual image capturing, and accordingly, a burden of the subject may be further reduced. A difference between a PAT apparatus of this embodiment and the PAT apparatus of the first embodiment will be mainly described hereinafter.

A configuration of the PAT apparatus of this embodiment is basically the same as that of the first embodiment illustrated in FIG. 1. However, operations of a condition obtaining unit 103, a state estimation unit 105, and a display controller 106 are different from those of the first embodiment. Other portions are the same as those of the first embodiment, and therefore, descriptions thereof are omitted.

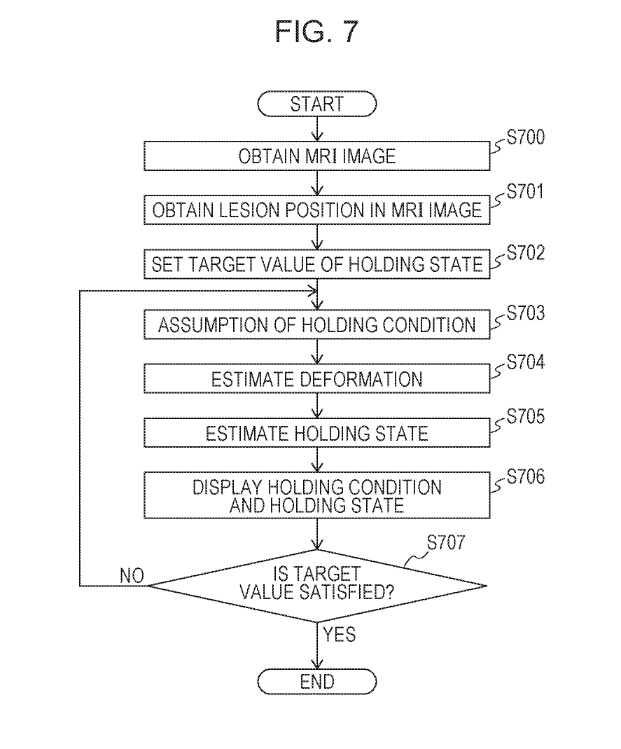

FIG. 7 is a flowchart illustrating a procedure of a process of estimating a holding state performed by a PAT apparatus 100 according to this embodiment. Note that operations in step S700, step S701, step S704, and step S705 are the same as the operations in step S300, step S301, step S303, and step S304 of the first embodiment illustrated in FIG. 3, and therefore, descriptions thereof are omitted. Hereinafter, only operations in step S702, step S703, step 3706, and step S707 will be described.

### [Step S702: Set Target Value]

In step S702, the state estimation unit 105 sets a target value of a holding state. In this embodiment, a target value of a distance between one of holding plates and a target portion is set. The target value may be determined in advance as a value (a reference value or an appropriate value) recommended by the PAT apparatus 100. Alternatively, a user may input the target value using an input apparatus, such as a keyboard, not illustrated. Hereinafter, an example of an operation will be described on the assumption that the user specifies 20 mm, for example, as the target value.

### [Step S703: Assumption of Holding Condition]

Next, in step S703, the condition obtaining unit 103 temporarily determines a holding length between the holding plates as a holding condition of a breast and sets the holding length as input information of a deformation estimation. For example, in a breast region 501 of an MRI image 500, a maximum value and a minimum value in a z-coordinate in an MRI image coordinate system 512 are obtained, and a value obtained by subtracting a predetermined value (20 mm, for example) from a difference between the maximum value and the minimum value is determined as a first value of the assumed holding length. Then every time the operation of this step is executed, the assumed holding length may be reduced by a predetermined value (5 mm, for example). Alternatively, a value input by the user using the input apparatus, such as a keyboard, may be set as the assumed holding length.

Thereafter, in step S704 and step S705, pressure deformation is estimated using the assumed holding length 505, and a holding-plate-to-target-portion distance 511 under the assumption is estimated.

### [Step S706: Display Holding Condition and Holding State]

In step S706, the display controller 106 displays information on the holding condition of the subject assumed in step S703, information on the holding state of the target portion estimated in step S705, and a breast-position-setting-camera image in a display not illustrated.

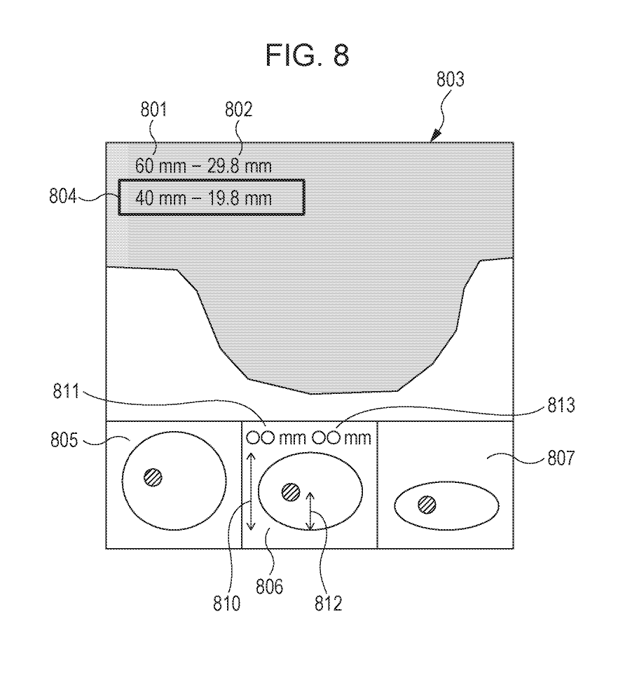

FIG. 8 is a diagram illustrating a screen 803 displayed in a case where the holding length is displayed as the holding condition and the holding-plate-to-target-portion distance is displayed as the holding state according to this embodiment. A holding length 801 illustrated in FIG. 8 corresponds to the holding length assumed in step S703. A holding-plate-to-target-portion distance 802 corresponds to the holding-plate-to-target-portion distance estimated in step S705.

An MRI image 805 is a cross-sectional image which is in parallel to a coronal surface and which includes the target portion in the obtained MRI image 500. A deformed MRI image 806 is a cross-sectional image which is in parallel to a coronal surface and which includes the target portion in a three-dimensional image obtained by performing a deformation process on the MRI image 500 in accordance with a result of the deformation estimation. According to FIG. 8, when the holding length assumed at first is 60 mm, the holding-plate-to-target-portion distance is estimated as 29.8 mm. An arrow mark 810 representing the holding length, a value 811 of the holding length (the holding condition), an arrow mark 812 representing the holding-plate-to-target-portion distance, and a value 813 of the holding-plate-to-target-portion distance (the holding state) are individually superposed on the deformed MRI image 806.

[Step 3707: Comparison with Target Value]

Next, in step S707, a calculation unit, not illustrated, included in the state estimation unit 105 compares the target value of the holding state set in step S702 with the holding state estimated in step S705. As a result of the comparison, when the holding state satisfies the target value, the display controller 106 informs a fact that the holding state satisfies the target value, and the estimation of the holding state is completed. As an informing method, a method for surrounding the holding length and the holding-plate-to-target-portion distance by a frame as illustrated by an informing display 804 of FIG. 8 may be employed as an example.

On the other hand, when the target value is not satisfied as a result of the comparison performed in step S707, the process returns to step S703 where the holding condition is assumed again and the process from step S703 onwards is performed. This process is repeatedly performed until the target value is satisfied. Then as a result of the repetitive process, a holding condition in which the holding state satisfies the target value is calculated as a recommendation value of the holding condition (a holding condition recommended by the apparatus), that is, an indication of the holding condition. According to FIG. 8, when 40 mm is set in a fifth setting after the holding length of 60 mm is set at first, the holding-plate-to-target-portion distance is estimated as 19.8 mm, and the distance satisfies the target value. A deformed MRI image in this case corresponds to a deformed MRI image 807 of FIG. 8.

As a method for displaying the holding condition (the holding length 801) and the holding state (the holding-plate-to-target-portion distance 802), a display method different from the display method in a list format may be employed as long as the relationship between the holding condition and the holding state which are associated with each other may be displayed. For example, a display method of a graph format may be employed. In this case, the holding condition (the holding length) is set in an axis of abscissa of the graph and the holding state (the holding-plate-to-target-portion distance) is set in an axis of ordinate of the graph. Furthermore, information on the target value of the holding state (the holding-plate-to-target-portion distance 802) is displayed on the graph. By this, an indication of the holding condition (the holding length) in which the holding state attains the target value may be easily recognized.

As described above, the PAT apparatus of this embodiment first sets the target value of the holding state to be estimated and the estimation of the holding state is repeatedly performed until the target value is satisfied. Accordingly, the user may recognize an indication of an appropriate holding condition before a breast is held.

### [Modification 2-1]

In the foregoing embodiment, the case where the holding-plate-to-target-portion distance is set as the target value of the holding state is described. However, any one of the other holding states described in the first embodiment may be set as the target value. Furthermore, a target value of stress in a position of the target portion may be set and a holding condition which satisfies the target value may be obtained.

### [Modification 2-2]

The information displayed in the process of step S706 may be replaced by other information. For example, only an appropriate holding condition and a corresponding holding state may be displayed without displaying holding states corresponding to holding conditions obtained every time calculation is performed. In this case, a smaller interval of the holding length may be set. Furthermore, the determination of the termination of the process may not be performed in step S707, and holding states corresponding to holding conditions in a predetermined range (for example, a holding length of 60 mm to 30 mm at an interval of 5 mm) may be obtained and displayed and thereafter an appropriate holding condition (which satisfied the target value and which corresponds to the maximum holding length) may be selected and displayed. Furthermore, the holding states corresponding to the holding conditions in the predetermined range may be calculated and displayed (for the user who performs a determination with reference to values of the holding states) without setting the target value of the holding state in the apparatus.

### Third Embodiment

The PAT apparatus according to the foregoing embodiments sandwiches the breast using the two holding plates. However, other holding methods for holding a subject may be employed. The PAT apparatus of a third embodiment presses a single holding plate against a breast from a nipple side so that the breast is held in a state in which a thickness of the breast is reduced. As a holding state, a target value of a holding-plate-to-target-portion distance is set and a holding condition which satisfies the target value, that is, a distance of movement of the holding plate, is calculated in advance as an indication of the holding condition to be displayed. A user may set an appropriate holding condition in accordance with a result of the calculation.

A configuration of the PAT apparatus of this embodiment is basically the same as that of the first embodiment illustrated in FIG. 1. However, a configuration of a holding unit 120 is different from that of the first embodiment. Furthermore, since a different method for holding a subject is employed, operations of a condition obtaining unit 103, a deformation estimation unit 104, and a state estimation unit 105 are different from those of the first embodiment. Other portions are the same as those of the first embodiment, and therefore, descriptions thereof are omitted.

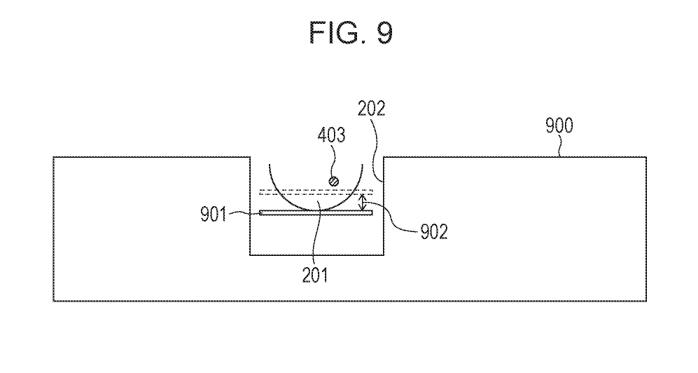

A configuration of the holding unit 120 of a PAT apparatus 900 of this embodiment will be described with reference to FIG. 9. As with the first embodiment, an examinee lies face down on a bed on the PAT apparatus 900. As illustrated in FIG. 9, a breast 201 is inserted into an opening portion 202. Here, the breast 201 is held by the holding unit 120 including a transparent holding plate 901 (a holding member) and image capturing is performed in a state in which a thickness of the breast 201 is reduced so that irradiation light reaches an inside of the breast 201. The breast 201 is held by moving the transparent holding plate 901 from a breast side to a back side. A distance of the movement of the transparent holding plate 901 (a holding-plate-movement distance 902) corresponds to a distance in which the transparent holding plate 901 moves from a position where the transparent holding plate 901 is in contact with the breast 201, that is, a position of a movement distance of 0, to a position where the transparent holding plate 901 arrives.

A procedure of a process of estimating a holding state by the PAT apparatus 900 of this embodiment is the same as that described with reference to the flowchart of the second embodiment illustrated in FIG. 7 except for operations in step S703, step S704, and step S705. Only these operations will be described hereinafter.

### [Step S703: Assumption of Holding Condition]

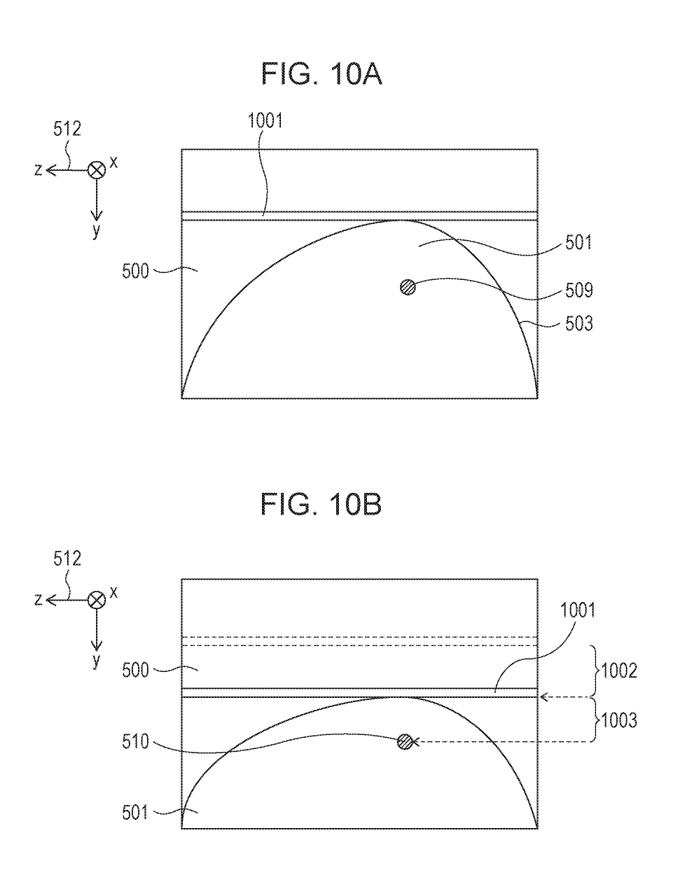

In step S703, as with the second embodiment, the condition obtaining unit 103 sets (assumes) a holding condition of a breast. As a concrete process in this embodiment, a distance of movement of the holding plate is assumed as a holding condition and is used as an input of a deformation simulation. Specifically, a y-coordinate in which a body surface 503 of a breast region 501 in an MRI image 500 is in contact with a holding plate 1001 in FIG. 10A is set as a movement distance of 0. Here, an MRI image coordinate system 512 is used. Thereafter, every time the operation of this step is executed, a holding-plate movement distance 1002 illustrated in FIG. 10B is increased by a predetermined value so that a movement distance of the holding plate is set.

### [Step S704: Deformation Estimation]

In step S704, as with the second embodiment, the deformation estimation unit 104 estimates deformation of a breast region included in the MRI image obtained in step S700 under the holding condition assumed in step S703. As a concrete operation in this embodiment, pressure deformation is estimated using the holding-plate movement distance 1002 assumed in step S703. The deformation estimation of this embodiment may be performed similarly to the first embodiment when the holding length used in the first embodiment is replaced by the holding-plate movement distance.

### [Step S705: Estimate Holding State]

In step S705, the state estimation unit 105 performs a process the same as that of the first embodiment so as to estimate a holding-plate-to-target-portion distance 1003 on the assumption of step S703.

As described above, even when a different method for holding a subject is employed, an indication of the holding condition which satisfies the target value of the holding state may be displayed.

### Fourth Embodiment

The PAT apparatuses of the foregoing embodiments employ a method for moving the position of the holding plate serving as a unit for adjusting the holding condition. However, the unit for adjusting the holding condition is not limited to the movement of the position of the holding plate. A PAT apparatus of a fourth embodiment uses a container (referred to as a “holding container”) having a basin shape as a holding member and adjusts a holding condition by selecting an appropriate container from among holding containers of different sizes depending on a size of a breast of a subject. Here, holding states (holding-container-surface-to-target-portion distances, for example) of a target portion obtained when the individual holding containers are used are estimated and displayed for a user. A user may select an appropriate holding container in accordance with a result of the estimation.

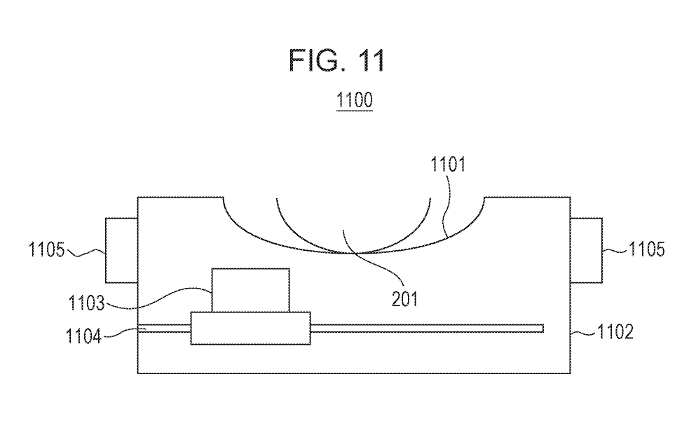

A configuration of a holding unit 120 of a PAT apparatus 1100 of this embodiment will be described with reference to FIG. 11. As with the first embodiment, an examinee lies face down on a bed on the PAT apparatus 1100. As illustrated in FIG. 11, a breast 201 is accommodated in a holding container 1101. The holding container 1101 is filled with water so that the breast 201 is soaked in the water. A case 1102 is filled with acoustic matching liquid. Furthermore, the case 1102 includes a prober unit 1103 which receives ultrasonic waves generated by irradiating light to a subject. A scanning mechanism 1104 is controlled to move a position of the prober unit 1103 so that an image of the entire breast 201 is captured. Here, the breast 201 is held in a state in which the breast 201 is pressed by the holding unit 120 including the holding container 1101 (the holding member) and image capturing is performed in a state in which a thickness of the breast 201 is reduced so that irradiation light reaches an inside of the breast 201. The holding container 1101 is selected from among a plurality of holding containers having different sizes, and the user manually sets a selected one of the holding containers having different sizes. It is assumed here that the PAT apparatus 1100 stores three-dimensional shape data of the holding containers in a storage region, not illustrated.

A procedure of a process of estimating a holding state by the PAT apparatus 1100 of this embodiment is the same as that described with reference to the flowchart of the second embodiment illustrated in FIG. 7 except for operations in step S703, step S704, and step S705. Only these processes will be described hereinafter.

### [Step S703: Assumption of Holding Condition]

In step S703, as with the foregoing embodiment, a condition obtaining unit 103 sets (assumes) a holding condition of a breast. As a concrete process in this embodiment, each of types of holding container is sequentially set as a holding condition in descending order of size. Specifically, every time this process is executed, a holding container of the smaller size is sequentially set. Then three-dimensional shape data of the selected container is set as input information of a deformation simulation.

### [Step S704: Deformation Estimation]

In step S704, as with the foregoing embodiment, a deformation estimation unit 104 estimates deformation of a breast region included in an MRI image obtained in step S700 under the holding condition assumed in step S703. As a concrete process of this embodiment, a deformation state at a time when the subject is held by the holding container is simulated in accordance with the three-dimensional shape of the holding container obtained in step S703. A result of the simulation is output to a state estimation unit 105.

### [Step S705: Estimation of Holding State]

In step S705, the state estimation unit 105 estimates the holding-container-surface-to-target-portion distance under the current holding condition in accordance with a result of the deformation simulation calculated in step S704. Specifically, first, a position of the target portion after the deformation is calculated similarly to the first embodiment. Then a shortest distance between the position (a three-dimensional coordinate) and the holding container (the three-dimensional shape data) is calculated, and this value is determined as an estimation value of the holding-container-surface-to-target-portion distance. The shortest distance between the three-dimensional coordinate and the three-dimensional shape model may be calculated by a general method.

As described above, even when a different method for adjusting a holding condition is employed, an indication of the holding condition which satisfies a target value of the holding state may be displayed.

### [Modification 4-1]

In the second to fourth embodiments, the holding plate(s) having a shape recognized in advance or a holding container is used to hold a subject. However, the subject holding members are not limited to rigid bodies, and stretchable holding films may be employed. In this case, as a holding condition assumed in step S703, at least one of a position of a holding film and tension of the holding film may be set. Furthermore, in a deformation estimation in step S704, deformation of a subject in an MRI image and the holding film are estimated such that a shape of the subject included in the MRI image coincides with a shape of the holding film in accordance with a shape of a body surface to which the holding film sticks taking the deformation of the holding film into consideration. Accordingly, the user may recognize an estimation of an appropriate holding condition of the holding film. Note that a function of estimating a holding state performed by an image obtaining unit 101, a position obtaining unit 102, a condition obtaining unit 103, a deformation estimation unit 104, a state estimation unit 105, and a display controller 106 may be implemented in an apparatus independently from the PAT apparatus.

### Fifth Embodiment

In the first embodiment, the holding state of the target portion, such as the distance between the target portion and one of the holding plates and stress applied to the target portion, is illustrated as a state of image capturing of the target portion. However, the state to be estimated is not limited to the holding state of the target portion, and any other state of image capturing of the target portion may be estimated as long as the state is obtained from a holding condition of a subject. An imaging apparatus according to a fifth embodiment estimates and displays an amount of reached irradiation light (reached light amount) in a position of a target portion and an attenuation rate of a signal at a time when a reception unit receives a photoacoustic signal generated in the same position as a state of image capturing of the target portion other than a holding state. Furthermore, reception strength and an SN ratio of the photoacoustic signal from the same position obtained from the information are estimated and displayed. A difference between a PAT apparatus of this embodiment which is an example of the imaging apparatus and the PAT apparatus of the first embodiment will be mainly described.

A configuration of the PAT apparatus of this embodiment is basically the same as that of the first embodiment illustrated in FIG. 1. However, operations of a state estimation unit 105 and a display controller 106 are different from those of the first embodiment. Other portions are the same as those of the first embodiment, and therefore, descriptions thereof are omitted.

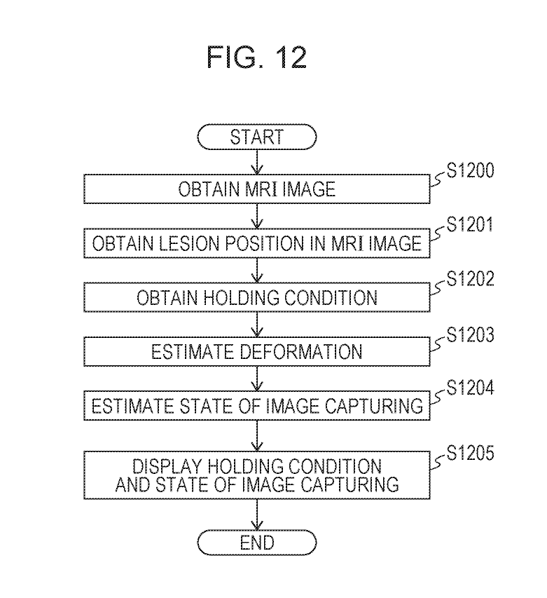

FIG. 12 is a flowchart illustrating a procedure of a process of estimating a state of image capturing performed by a PAT apparatus 100 according to this embodiment. Note that operations in step S1200, step S1201, step S1202, and step S1203 are the same as the operations in step S300, step S301, step S302, and step S303 of the first embodiment illustrated in FIG. 3, and therefore, descriptions thereof are omitted. Hereinafter, only operations in step S1204 and step S1205 will be described.

### [Step S1204: Estimate State of Image Capturing]

In step S1204, the state estimation unit 105 estimates information on a state of image capturing of a target portion under a current holding condition in accordance with a result of a deformation estimation of an MRI image calculated in step S1203. Specifically, an amount of light reached in the position of the target portion, an attenuation rate of a photoacoustic signal supplied from the same position (an amount of attenuation in a position of a reception unit 135), a reception intensity and an SN ratio of the photoacoustic signal supplied from the same position, and resolution and contrast of the signal which is generated as an image are estimated.

Here, the amount of light reached in the position of the target portion may be estimated in accordance with an estimated shape of the subject, the position of the target portion, a standard characteristic of a target object associated with light absorption, information (irradiation conditions including an irradiation position, an irradiation intensity, and the number of times irradiation is performed) on light irradiated by an irradiation unit 130, and the like. For example, the amount of reached light is obtained by multiplying an amount of light which is incident on the subject by exp(−(an average equivalent attenuation coefficient of the subject)·(a distance between a surface of the subject and the target portion)). Furthermore, the amount of reached light is obtained by a general light distribution simulation taking a shape of the subject and profile of the incident light into consideration. The attenuation rate of the photoacoustic signal supplied from the same position may be estimated in accordance with the estimated shape of the subject, the position of the target portion, the standard characteristic of the target object associated with ultrasonic waves, information on the position of the reception unit 135, and the like. Furthermore, the reception intensity and the SN ratio of the photoacoustic signal and the resolution and the contrast at a time when an image of the signal is generated may be estimated in accordance with the amount of reached light estimated as described above, the attenuation rate of the photoacoustic signal, and a standard light absorption characteristic (an absorption coefficient) of the target portion (a blood vessel, for example). Since the calculations of the values are generally recognized as simulation techniques of a photoacoustic tomography, detailed descriptions thereof are omitted.

### [Step S1205: Display Holding Condition and State of Image Capturing]

In step S1205, a display controller 106 displays a current holding condition of the subject measured in step S1202, the states of image capturing estimated in step S1204, and an image captured by a breast-position-setting camera 205 in a display, not illustrated. Here, all the states estimated in step S1304 may be displayed or only a state specified by a user through a UI, not illustrated, may be displayed.

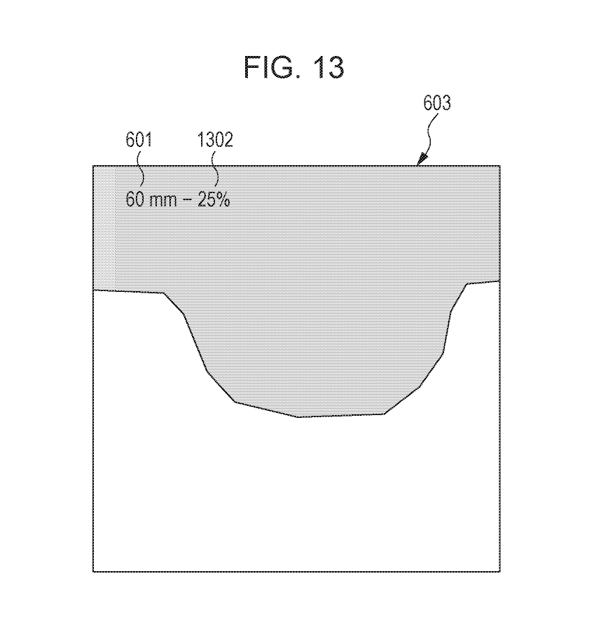

FIG. 13 is a diagram illustrating a screen displayed in a case where an attenuation rate of a photoacoustic signal is displayed as the state of image capturing of the target portion according to this embodiment. Here, unlike the example of the screen in the first embodiment illustrated in FIG. 6, an attenuation rate 1302 of a photoacoustic signal is displayed instead of the holding state 602. According to the example of FIG. 13, it is estimated that a current holding length is 60 mm and an attenuation rate of the photoacoustic signal is 25 percent. Note that the attenuation rate of the photoacoustic signal represents that the signal is not attenuated when the attenuation rate is 0 percent and the signal is attenuated to zero when the attenuation rate is 100 percent.

As described above, according to the imaging apparatus of this embodiment, the user may adjust the holding condition while determining whether the state of image capturing of the target portion is appropriate in accordance with the displayed information and capture a PAT image.

Note that the information to be displayed may be a rate of light reached in the target portion instead of the amount of reached light. Furthermore, instead of the attenuation rate of the acoustic signal, a reaching rate may be employed. Moreover, the information on the holding state described in the first embodiment may be simultaneously displayed.

### Sixth Embodiment

In the second embodiment, the holding states of the target portion corresponding to the plurality of assumed holding conditions are estimated in advance, and an indication of the holding conditions is calculated in accordance with the target value of the holding states, and displayed for the user. On the other hand, an imaging apparatus according to a sixth embodiment obtains assumption of an image capturing condition in addition to a holding condition, estimates a state of image capturing of a target portion in accordance with a combination of the holding condition and the image capturing condition, and the estimated state is displayed. Then an indication of the combination between a holding condition and an image capturing condition is calculated in accordance with a target value of the state of image capturing to be displayed for a user. A difference between a PAT apparatus of this embodiment which is an example of an imaging apparatus and the PAT apparatus of the second embodiment will be mainly described.

A configuration of the PAT apparatus of this embodiment is basically the same as that of the second embodiment. However, operations of a condition obtaining unit 103, a state estimation unit 105, and a display controller 106 are different from those of the second embodiment. Other portions are the same as those of the second embodiment, and therefore, descriptions thereof are omitted.

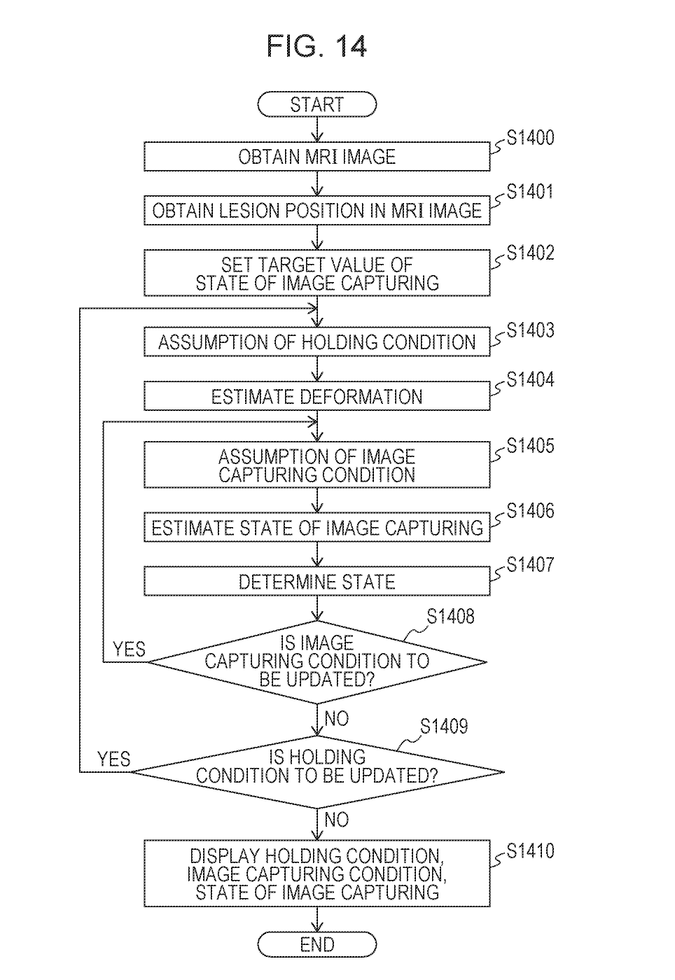

FIG. 14 is a flowchart illustrating a procedure of a process of estimating a state of image capturing and displaying the state by a PAT apparatus 100 according to this embodiment. Note that operations in step S1400, step S1401, step S1403, and step S1404 are the same as the operations in step S700, step S701, step S703, and step S704 of the second embodiment illustrated in FIG. 7, and therefore, descriptions thereof are omitted. Hereinafter, only an operation in step S1402 and operations in step S1405 to step S1409 will be described.

### [Step 31402: Set Target Value of State of Image Capturing]

In step S1402, a state estimation unit 105 sets a target value of a state of image capturing. For example, a target value of an amount of irradiation light reached in the target portion (an amount of reached light) is set. The target value may be determined in advance as a value (a reference value or an appropriate value) recommended by the PAT apparatus 100. Furthermore, a user may input the target value using an input apparatus, such as a keyboard, not illustrated. Hereinafter, an operation performed on the assumption that 50 mJ/mm², for example, is set as a reference value will be described as an example.

### [Step S1405: Assumption of Image Capturing Condition]

Next, in step S1405, a condition obtaining unit 103 assumes an image capturing condition of a subject as input information for estimating the state of image capturing. An irradiation condition (an irradiation position, an irradiation intensity, the number of times irradiation is performed, and the like) of irradiation light irradiated by the irradiation unit 130 is an image capturing condition of the subject. In this embodiment, the number of times irradiation is performed among the irradiation conditions described above is assumed as a variable condition. The number of times irradiation is performed is associated with resolution and contrast of a captured image. The larger the number of times irradiation is performed is, the higher the resolution and the contrast of the image are. Furthermore, since the number of times irradiation is performed per second of irradiation pulse light is fixed (for example, 10 Hz or 20 Hz), an image capturing time required for one image capturing which is calculated from the number of times irradiation is performed may be assumed as an image capturing condition.

Here, a value (30, for example) determined in advance in accordance with a standard characteristic of the subject and performance of a light source is used as an initial value of the number of times irradiation is performed. Furthermore, a user may input the initial value using an input apparatus, such as a keyboard. Every time the operation of this step is executed, the number of times irradiation is performed is increased while a holding length is fixed. The number of times irradiation is performed may be increased by a predetermined value (10, for example) or the user may input a value.

### [Step S1406: Estimate State of Image Capturing]

In step S1406, a state estimation unit 105 estimates the state of image capturing of the target portion and transmits information on the state to a display controller 106. A process of this step is the same as that in step S1204 of the fifth embodiment, and therefore, a detailed description thereof is omitted.

### [Step S1407: Determination of State]

In step S1407, a determination unit, not illustrated, included in the state estimation unit 105 compares the state estimated in step S1406 with a target value set in step S1402 so as to determine whether the estimated state satisfies the target value (a quality determination).

### [Step S1408: Is Image Capturing Condition to be Updated?]

In step S1408, the condition obtaining unit 103 determines whether an image capturing condition is to be updated in accordance with a result of a determination as to whether all image capturing conditions to be assumed have been assumed. For example, when the number of times irradiation is performed has reached an upper limit (60, for example), the condition obtaining unit 103 determines that the image capturing condition is not to be updated and the process proceeds to step S1409. On the other hand, when the number of times irradiation is performed has not reached the upper limit, the condition obtaining unit 103 determines that the image capturing condition is to be updated and the process returns to step S1405. After the image capturing condition is updated, the operation in step S1406 is executed again.

### [Step S1409: Is Holding Condition to be Updated?]

In step S1409, the condition obtaining unit 103 determines whether a holding condition is to be updated in accordance with the result of the determination as to whether all holding conditions to be assumed have been assumed. For example, when a holding length has reached a lower limit (40 mm, for example), the condition obtaining unit 103 determines that the holding condition is not to be updated and the process proceeds to step S1410. On the other hand, when the holding length has not reached the lower limit, the condition obtaining unit 103 determines that the holding condition is to be updated and the process returns to step S1403. After the holding condition is updated, the operation in step S1404 onwards is executed again.

By the process described above, states of image capturing of the target portion corresponding to combinations of the holding conditions and the image capturing conditions are estimated. Furthermore, as a result of the repetitive process, a combination of a holding condition and an image capturing condition in which the state of image capturing of the target portion satisfies the target value is calculated as a recommendation value of the holding condition and the image capturing condition (a combination of a holding condition and an image capturing condition recommended by the apparatus).

### [Step 31410: Display Holding Condition, Image Capturing Condition, and State of Image Capturing]

In step S1410, a display controller 106 displays the holding condition assumed in step S1403, the image capturing condition assumed in step S1405, and the state of image capturing of the target portion estimated in step S1406 in a display, not illustrated. Furthermore, the display controller 106 displays a result of the quality determination of the state of image capturing determined in step S1407 in the display, not illustrated.

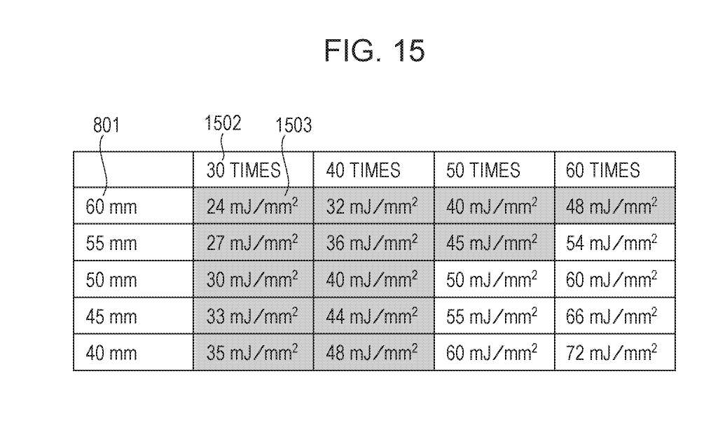

FIG. 15 is a diagram illustrating a screen displayed in a case where the holding length which is the holding condition, the number of times irradiation of light is performed which is the image capturing condition, and an amount of irradiation light reached in the position of the target portion estimated as the state of image capturing are displayed in this embodiment. A holding length 801 illustrated in FIG. 15 corresponds to a holding length assumed in step S1403. A number of times irradiation is performed 1502 corresponds to the number of times irradiation is performed assumed in in step S1405. An amount of reached irradiation light 1503 corresponds to the amount of irradiation light reached in the position of the target portion estimated in step S1406. As illustrated, in this embodiment, results of estimations are displayed as a list in a format of a table including holding conditions in rows and image capturing conditions in columns. According to the example of FIG. 15, assuming that the holding length is 60 mm and the number of times irradiation is performed is 30, the amount of reached irradiation light is estimated as 24 mJ/mm². Furthermore, combinations which do not satisfy the target value of the amount of reached irradiation light are displayed by gray, and by this, quality of the state is displayed. Furthermore, the state of image capturing of the target portion (including an attenuation amount of a photoacoustic signal) may be displayed instead of the amount of reached irradiation light described in the fifth embodiment. Moreover, target values of a plurality of conditions may be set and results of determinations as to whether the target values are satisfied may be displayed. A result of a determination as to whether all the conditions are satisfied may be displayed. As for the image capturing condition, a condition other than the number of times irradiation is performed may be displayed. For example, a period of time image capturing is performed defined by the number of times irradiation is performed may be displayed.

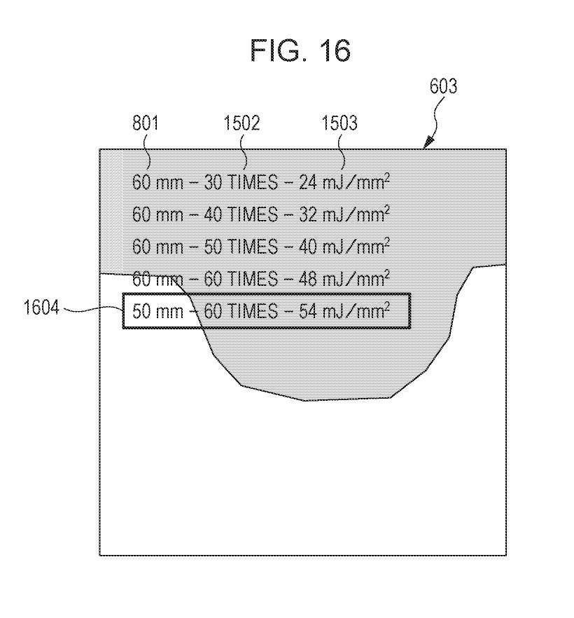

Note that, instead of the display of all the combinations of the conditions described above, only combinations of the conditions which satisfy a condition may be displayed in a list form. Furthermore, as with the second embodiment, the process may be interrupted when a condition which satisfies the target value is detected and the condition may be displayed. In this case, priority levels are assigned to the holding condition and the image capturing condition and the holding condition may be changed only when the target value is not reached even though the image capturing condition is changed. In this case, display illustrated in FIG. 16 may be performed, for example. According to the example illustrated in FIG. 16, even when the holding length of 60 mm is set first and the number of times irradiation is performed of 60 which is an upper limit is set, the amount of reached irradiation light does not satisfy the target value. In this case, when the holding length is changed to 50 mm and the number of times irradiation is performed of 60 is not changed, the amount of reached irradiation light attains the target value and an indication 1604 representing that the amount of reached irradiation light attains the target value is displayed.

As described above, the PAT apparatus according to this embodiment estimates the state of image capturing in accordance with the assumption of the holding condition and the image capturing condition. Accordingly, the user may recognize indications of an appropriate holding condition and an appropriate image capturing condition before the subject is held.

### [Modification 6-1]

In the foregoing embodiment, the case where recommendation values of the holding condition and the image capturing condition are calculated after the holding condition and the image capturing condition are both assumed has been described. However, only a recommendation value of one of the holding condition and the image capturing condition may be calculated. When an image of the same examinee is captured in different days, for example, the same holding length may be set so that substantially the same breast shape is obtained. In such a case, the operation in step S1409 in the flowchart of FIG. 14 is not required. Accordingly, the user may calculate only a recommendation value of the image capturing condition while using a predetermined value as the holding condition. Furthermore, when a period of time in which image capturing is performed is limited, the image capturing condition is fixed, and only the holding condition may be estimated under the limitation.

### [Modification 6-2]

In the foregoing embodiment, the combinations of the holding conditions and the image capturing conditions are estimated before the subject is held, and the states of image capturing of the target portion relative to the combinations are estimated. However, various image capturing conditions may be assumed under a holding condition which is an actual holding condition in the state in which the subject is held, and the states of image capturing of the target portion relative to the various image capturing conditions may be displayed. Accordingly, the user may recognize an appropriate image capturing condition in a current holding state. The user may determine whether the condition of image capturing satisfies the target value by changing the image capturing condition, and in accordance with information on the determination, the user may determine whether the holding condition is to be further adjusted.

### [Modification 6-3]

In the foregoing embodiment, the number of times irradiation is performed on the target portion is calculated as the image capturing condition. However, when image capturing is performed using a PAT apparatus by scanning a subject, only the required number of times irradiation to the target portion is performed (the number of times irradiation is performed which is effective for the target portion) is determined. Accordingly, in this case, an image capturing parameter of the entire subject may be calculated from the number of times irradiation is performed on the target portion.

### Other Embodiments

The present invention is also realized by executing the following process. Specifically, software (programs) which realizes the functions of the foregoing embodiments are supplied to a system or an apparatus through a network or various storage media, and a computer (or a CPU, an MPU, or the like) of the system or the apparatus reads and executes the programs.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-230863, filed Nov. 13, 2014 and No. 2014-014755, filed Jan. 29, 2014, which are hereby incorporated by reference herein in their entirety.

### REFERENCE SIGNS LIST

101 Image obtaining unit
102 Position obtaining unit
103 Condition obtaining unit
104 Deformation estimation unit
105 State estimation unit

1. An imaging apparatus, comprising:
an image obtaining unit configured to obtain a three-dimensional image of a subject;
a target portion obtaining unit configured to obtain positional information of a target portion of the three-dimensional image obtained by the image obtaining unit;
a condition obtaining unit configured to obtain a holding condition for holding the subject by a holding member;
a deformation estimation unit configured to estimate deformation of the subject in the three-dimensional image in accordance with the holding condition obtained by the condition obtaining unit; and
a state estimation unit configured to estimate a state of image capturing of the target portion in accordance with the positional information of the target portion obtained by the target portion obtaining unit and the deformation of the subject estimated by the deformation estimation unit.

2. The imaging apparatus according to claim 1, wherein
the state of image capturing of the target portion estimated by the state estimation unit corresponds to the holding state of the target portion.

3. The imaging apparatus according to claim 2, wherein
the holding state of the target portion estimated by the state estimation unit includes a distance between the target portion and the holding member or a body surface of the subject.

4. The imaging apparatus according to claim 2, wherein
the holding state of the target portion estimated by the state estimation unit includes stress applied to the target portion.

5. The imaging apparatus according to claim 1, further comprising:
a reception unit configured to receive an acoustic signal supplied from the subject,
wherein the state of image capturing of the target portion estimated by the state estimation unit corresponds to an attenuation rate of the acoustic signal which is generated by the target portion and which is received by the reception unit.

6. The imaging apparatus according to claim 1, wherein
the condition obtaining unit obtains the holding condition in a state in which the subject is held by the holding member.

7. The imaging apparatus according to claim 1, further comprising:
a determination unit configured to perform a quality determination of the holding condition in accordance with the state of image capturing of the target portion estimated by the state estimation unit.

8. The imaging apparatus according to claim 1, further comprising:
a calculation unit configured to calculate a recommended holding condition in accordance with the state of image capturing of the target portion estimated by the state estimation unit.

9. The imaging apparatus according to claim 1, wherein
the condition obtaining unit obtains an image capturing condition of the subject, and
the state estimation unit estimates a state of image capturing of the target portion also in accordance with the image capturing condition obtained by the condition obtaining unit.

10. The imaging apparatus according to claim 9, further comprising:
an irradiation unit configured to irradiate near infrared light to the subject, wherein the image capturing condition corresponds to an irradiation condition of the irradiation unit.

11. The imaging apparatus according to claim 10, wherein
the irradiation condition corresponds to the number of time irradiation light is irradiated from the irradiation unit, a period of time image capturing is performed, and/or an irradiation intensity.

12. The imaging apparatus according to claim 10, wherein
the irradiation condition corresponds to an irradiation position of the irradiation unit.

13. The imaging apparatus according to claim 10, wherein
the state of image capturing of the target portion estimated by the state estimation unit corresponds to an amount of irradiation light irradiated by the irradiation unit and reached in the target portion.

14. The imaging apparatus according to claim 10, wherein
the state of image capturing of the target portion estimated by the state estimation unit corresponds to resolution of the target portion or contrast information.

15. The imaging apparatus according to claim 9, further comprising:
a determination unit configured to perform a quality determination of the holding condition and/or the image capturing condition in accordance with the state of image capturing of the target portion estimated by the state estimation unit.

16. The imaging apparatus according to claim 9, further comprising:
a calculation unit configured to calculate a recommended holding condition and/or an imaging condition in accordance with the state of image capturing of the target portion estimated by the state estimation unit.

17. An imaging method comprising:
obtaining a three-dimensional image of a subject;
obtaining positional information of a target portion of the obtained three-dimensional image;
obtaining a holding condition for holding the subject by a holding member;
estimating deformation of the subject in the three-dimensional image in accordance with the obtained holding condition; and
estimating a state of image capturing of the target portion in accordance with the obtained positional information of the target portion and the estimated deformation of the subject.

18. An imaging method comprising:
obtaining a three-dimensional image of a subject;
obtaining positional information of a target portion of the obtained three-dimensional image;
obtaining a holding condition for holding the subject by a holding member and an image capturing condition of the subject;
estimating deformation of the subject in the three-dimensional image in accordance with the obtained holding condition; and
estimating a state of image capturing of the target portion in accordance with the obtained positional information of the target portion, the estimated deformation of the subject, and the obtained image capturing condition.

19. A computer-readable storage medium storing a program that causes a computer to execute the processes in the imaging method disclosed in claim 17.

20. A computer-readable storage medium storing a program that causes a computer to execute the processes in the imaging method disclosed in claim 18.
